(19) United States
(12) Patent Application Publication
(10) Pub. No.: US 2009/0069441 A1
(43) Pub. Date: Mar. 12, 2009

(54) Motuporamine Mimic Agents

(76) Inventor: Otto Phanstiel, Oviedo, FL (US)

Correspondence Address: Beusse Wolter Sanks Mora & Maire, 390 N. ORANGE AVENUE, SUITE 2500, ORLANDO, FL, 32801, US

(21) Appl. No.: 12/037,120

(22) Filed: Feb. 26, 2008

Related U.S. Application Data
(63) Continuation-in-part of application No. 11/371,945, filed on Mar. 14, 2006.
(60) Provisional application No. 60/781,963, filed on Mar. 13, 2006.

(51) Int. Cl. A61K 31/137; C07C 211/27; A61P 35/00

(52) U.S. Cl. 514/655; 564/387

(57) ABSTRACT

Disclosed herein are motuporamine mimic agents and methods of making and using same. Particularly exemplified are motuporamine mimic agents comprising cytotoxic activity and/or anti-metaplastic activity.

### CROSS-REFERENCE TO RELATED APPLICATIONS

This application is a continuation-in-part of U.S. Ser. No. 11/371,945 filed Mar. 14, 2006, which claims priority to U.S. Provisional Application of the same title and Applicant name filed Mar. 13, 2006 to which priority is claimed under 35 USC § 119(e).

### BACKGROUND

The nonselective delivery of drugs to both targeted tumor cells and healthy cells is a major shortcoming of current chemotherapies. Enhanced cell targeting during drug delivery could diminish nonspecific toxicities by reducing uptake by healthy cells. Using existing cellular transporters for drug delivery provides opportunities for molecular recognition events to assist in the cell targeting process.

Ever since the published report of the discovery of motuporamines (see 1-3 FIG. 1), naturally occurring anti-cancer agents, found off the coast (Motupore Island) of new Guinea, the molecular structure and their bio-functions have fascinated biochemists (Williams et al., J. Org Chem 1998, 63:4838:4841; Williams et al., J. Org. Chem. 2002, 67:245-248; Roskelley et al., Cancer Res. 2001, 61:6788-6794). Indeed, In light of the difficulty and expense of obtaining and purifying natural motuporamines, efforts have been made toward developing analogous compounds having similar or better characteristics that may be synthetically manufactured. Dihydromotuporamine C, (see 4a, FIG. 1) comprises a fifteen-membered ring, which is difficult to synthesize unless one uses expensive metal catalysts like Grubb's catalyst.

### BRIEF DESCRIPTION OF THE DRAWINGS

FIG. 1 shows structures of motuporamine mimic agents.

FIG. 2 shows a scheme for synthesizing motuporamine mimic agents.

FIG. 3. shows a scheme for synthesizing motuporamine mimic agents.

FIG. 4 shows a scheme for synthesizing motuporamine mimic agents.

FIG. 5 shows a scheme for synthesizing motuporamine mimic agents.

FIG. 6 shows a scheme for synthesizing motuporamine mimic agents.

FIG. 7 shows a scheme for synthesising motuporamine mimic agents.

FIG. 8 shows a chemical compound structure.

FIG. 9 shows a chemical compound structure.

### DETAILED DESCRIPTION

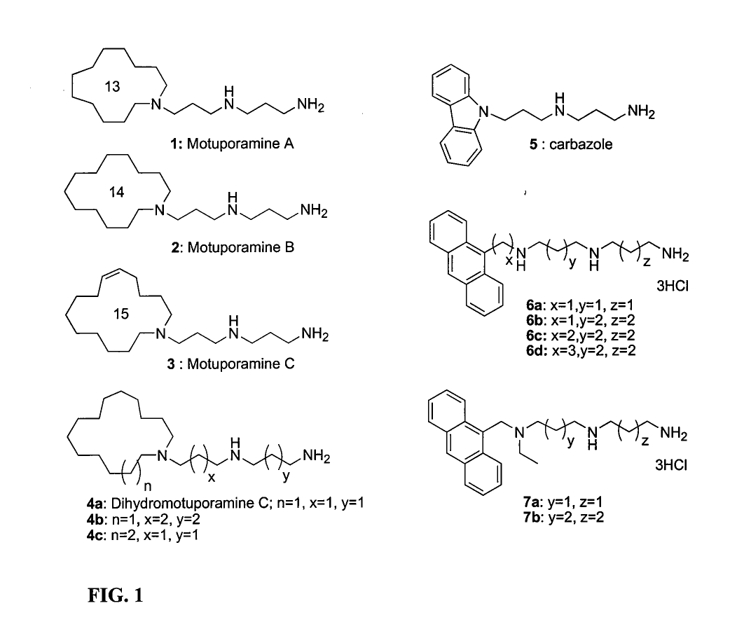

The invention pertains to motuporamine mimic agents (MMA) and synthesis thereof that are cytotoxic to cancer cells and optionally also inhibit their spread to other tissues (i.e., their metastatic behavior). The invention is based in part on the inventors' realization that less costly and easily synthesized motuporamine mimic agents are needed and desired. According to one aspect, the subject invention pertains to MMAs that have similar biological potency as compound 4a (see FIG. 1), yet are much easier to synthesize and provide a cost-efficient entry into this novel drug class. It is difficult chemically to synthesize 4a. There are several reports (Goldring, W. P. D.; Weiler, L. Cytotoxic Alkaloids Motuporamines A-C: Synthesis and Structural Verification, Org. Letters 1999, 1(9); 1471-1473; Furstner, A.; Rumbo, A. Ring-Closing Alkyne Metathesis. Stereoselective Synthesis of the Cytotoxic Marine Alkaloid Motuporamine C, J. Org. Chem. 2000, 65(8); 2608-2611), which synthesize 4a through lengthy synthesis steps involving expensive metal catalysts.

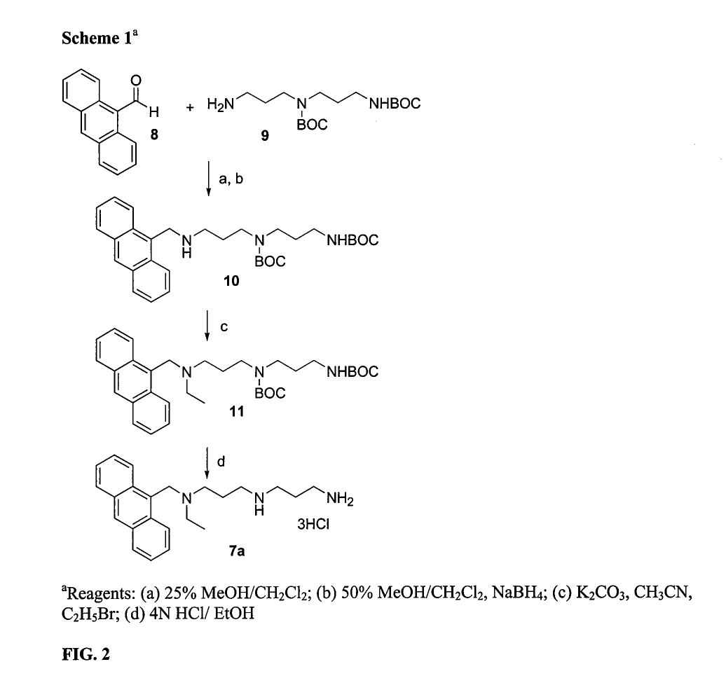

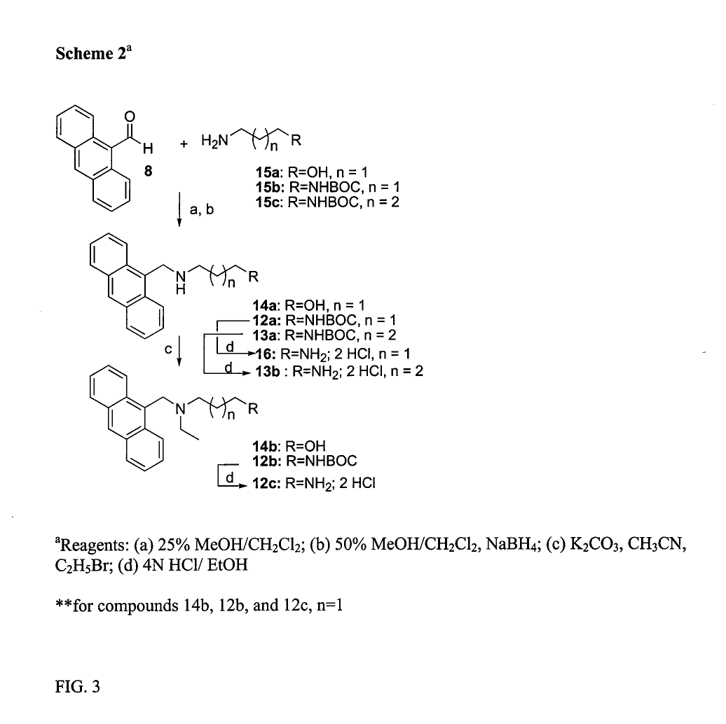

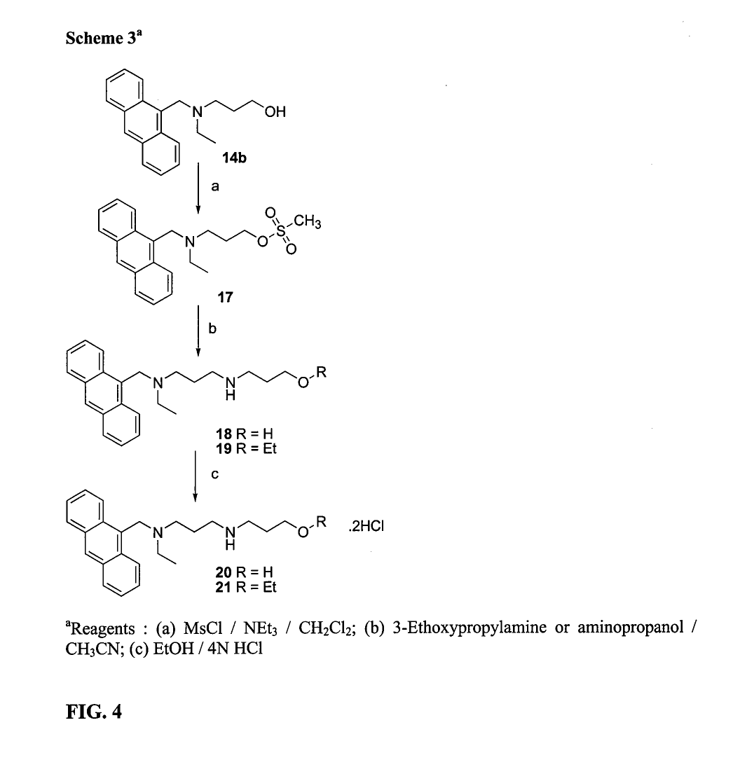

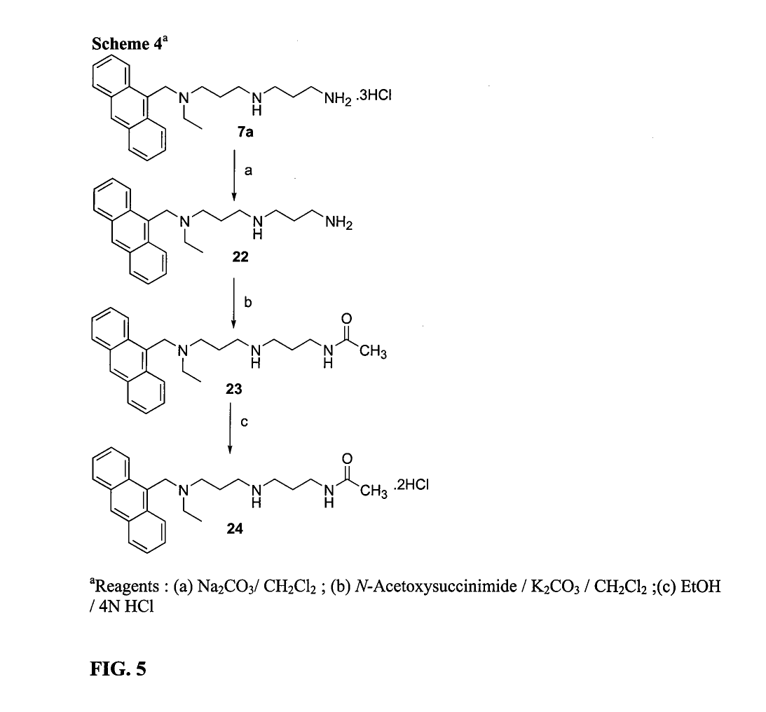

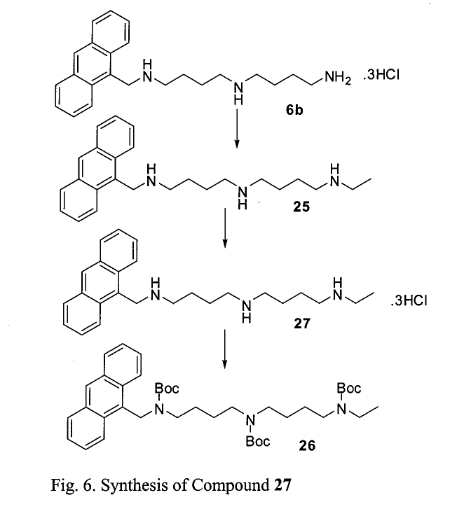

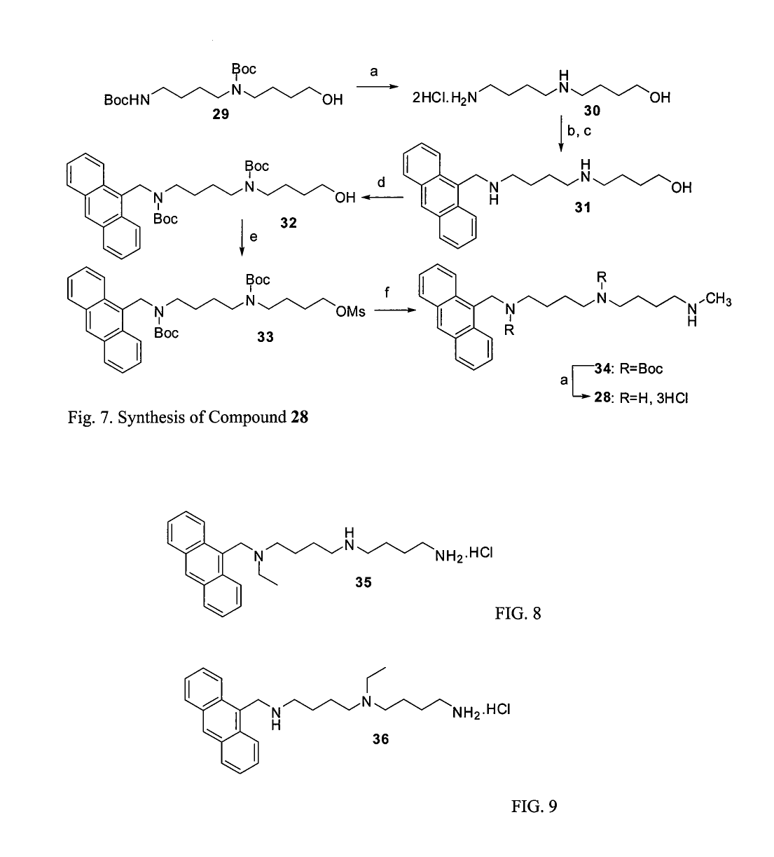

Certain MMA embodiments; of the subject invention such as, but not limited to, compounds 7a, 7b, 12 and 14, are more readily synthesized via synthesis schemes of the subject invention, including schemes 1 and 2 shown in FIGS. 2 and 3, respectively. Not being held to any particular theory, it is the inventors belief that the large anthracene ring system can substitute (and behave biologically) like the 15-membered ring of 4a. Alternate embodiments of the subject invention include, but are not limited to, 14b 17, 18, 19, 20, 21, as synthesized according to scheme 3 (FIG. 4) and compounds 22, 23, and 24 as synthesized according to scheme 4 (FIG. 5. Furthermore, other embodiments of the subject invention pertain to the synthesis processes disclosed in FIGS. 2-5, or portions thereof.

Certain MMA embodiments of the subject invention, such as, but not limited to 7a, 7b, 12 and 14 not only are good anticancer agents via their cytotoxic properties, but they also serve as anti-metastatic agents which block the spread of cancer cells (a common problem encountered with cancer patients). A non-toxic anti-metastatic agent would also be of use to cancer patients because it could be taken as a cancer preventative and/or as an anti-metastatic agent along with a different chemotherapeutic regimen. Accordingly, cytotoxic agents like 7a are helpful toward halting the spread of cancer as well as killing cancer cells.

In a specific embodiment, MMAs according to the subject invention comprise the following structure:

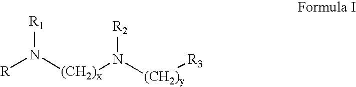

where R is alkylaryl (wherein the aryl ring is either a benzene, naphthalene, anthracene or pyrene ring system and the alkyl chain length is either methylene, ethylene, propylene, butylene, pentylene or hexylene), alkyl, cycloalkyl;

R₁ is either hydrogen or linear alkyl (methyl, ethyl, propyl, butyl, pentyl or hexyl) or branched alkyl (isopropyl, isobutyl, sec-butyl or t-butyl), or alkylaryl (wherein the aryl ring is either a benzene, naphthalene, anthracene or pyrene ring system and the alkyl chain length is either methylene, ethylene, propylene, butylene, pentylene or hexylene),

R₂ is either hydrogen, alkyl, alkylaryl or aryl or equivalent to the —(CH2)yR₃

R₃ is either hydrogen (H), or hydroxy (—OH), or alkoxy (—O-alkyl) or alkylamido (—NHCOalkyl), amino (—NH2) or aminoalkyl (—NH-alkyl), or N-alkyl, N-alkylamido, or Nalkylaryl,N-alkyl amino, x=1-16 and y=1-16 and pharmaceutically relevant inorganic salts thereof. MMA agents include pharmaceutically acceptable inorganic salts of the MMA agents (e.g., trihydrochloride salt, 3HCl salt of skeleton 1). Other embodiments of the subject invention pertain to methods of synthesizing MMA agents.

Certain preferred MMA embodiments include the following:

1: R=anthracen-9-ylmethyl, R₁=ethyl, R₂═H, R₃═NH₂, x=3, and y=3

2: R=anthracen-9-ylmethyl, R₁=ethyl, R₂═H, R₃═NH₂, x=4, and y=4

3: R=anthracen-9-ylmethyl, R₁=ethyl, R₂═H, R₃═OH, x=3, and y=3

4: R=anthracen-9-ylmethyl, R₁=ethyl, R₂═H, R₃═NHCOCH₃, x=3, and y=3

5: R=anthracen-9-ylmethyl, R₁=ethyl, R₂═H, R₃═OCH₂CH₃, x=3, and y=3

6: R=anthracen-9-ylmethyl, R₁═H, R₂═H, R₃═NH₂, x=3, and y=3

7: R=anthracen-9-ylmethyl, R₁═H, R₂═BOC, R₃═NHBOC, x=3, and y=3

8: R=anthracen-9-ylmethyl, R₁=ethyl, R₂═BOC, R₃═NHBOC, x=3, and y=3

9: R=anthracen-9-ylmethyl, R₁═H, R₂═BOC, R₃═NHBOC, x=4, and y=4

10: R=anthracen-9-ylmethyl, R₁=ethyl, R₂═BOC, R₃═NHBOC, x=4, and y=4

11: R=anthracen-9-ylmethyl, R₁=ethyl, R₂═H, R₃═NHCOR₄, where R₄ is linear or branched alkyl, aryl or alkylaryl, x=3, and y=3

In an alternative embodiment, MMAs according to the subject invention comprise the following structure:

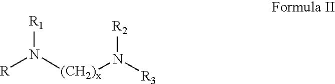

13: R=anthracen-9-ylmethyl, R₁=ethyl, R₂═H, R₃═H, x=3

14: R=anthracen-9-ylmethyl, R₁=ethyl, R₂═H, R₃═BOC (t-butylcarbonyloxy), x=3

15: R=anthracen-9-ylmethyl, R₁═H, R₂═H, R₃═H, x=3

16: R=anthracen-9-ylmethyl, R₁═H, R₂═H, R₃═BOC (t-butylcarbonyloxy), x=3

In an alternative embodiment, MMAs according to the subject invention comprise the following structure:

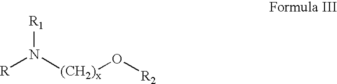

17: R=anthracen-9-ylmethyl, R₁=ethyl, R₂═H, x=3

18: R=anthracen-9-ylmethyl, R₁═H, R₂═H, x=3

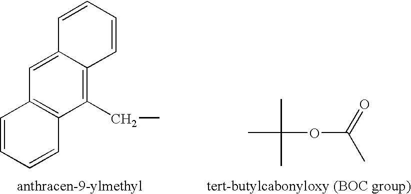

The following structures are those referred to in the formulas above:

Formula IV Formula V

### Pharmaceutical Compositions

The invention also pertains to pharmaceutical compositions which can be administered to a patient to achieve a therapeutic effect, e.g., cytotoxicity of cancer cells in a subject and/or metastatic behavior. Pharmaceutical compositions of the invention can comprise, for example, a Motuporamine Mimic Agent (MMA). The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically acceptable carriers comprising excipients and auxiliaries which facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethylcellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations which can be used orally include push fit capsules made of gelatin, as well as soft, sealed capsules made of gelatin and a coating, such as glycerol or sorbitol. Push fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 150 mM histidine, 0.1%2% sucrose, and 27% mannitol, at a pH range of 4.5 to 5.5, that is combined with buffer prior to use.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung, liver, spleen, heart brain, lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 10⁶ cells, more preferably about 1.0 μg of DNA per 16 nmole of liposome delivered to about 10⁶ cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

Determination of a Therapeutically Effective Dose

The determination of a therapeutically effective dose is well within the capability of those skilled in the art. A therapeutically effective dose refers to that amount of active ingredient which causes cytotoxicity of cancer cells in a subject and/or metastatic behavior which occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of container and labeled for treatment of an indicated condition. Such labeling would include amount, frequency, and method of administration.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, mammals such as dogs, cats, cows, horses, rabbits, monkeys, and most preferably, humans.

### EXAMPLE 1

### Synthesis of N-(3-Amino-propyl)-N′-anthracen-9-ylmethyl-N′-ethyl-propane-1,3-diamine, Hydrochloride salt (7a)

Materials. Materials for Examples 1-16. Silica gel (32-63 μm) and chemical reagents were purchased from commercial sources and used without further purification. All solvents were distilled prior to use. All other reactions were carried out under an N₂ atmosphere. ¹H and ¹³C spectra were recorded at 300 or 75 MHz, respectively. TLC solvent systems are listed as volume percents and NH₄OH refers to concentrated aqueous NH₄OH.

A solution of BOC-protected 11 (700 mg, 1.28 mmole) was dissolved in absolute ethanol (13 mL) and stirred at 0° C. for 10 minutes. A 4N HCl solution (22 mL) was added to the reaction mixture dropwise and stirred at 0° C. for 20 minutes and then at room temperature overnight. The solution was concentrated in vacuo to give 7a as a yellow solid in 90% yield. ¹H NMR (D₂O) δ 8.23 (s, 1H), 7.91 (m, 4H), 7.70 (m, 2H), 7.59 (m, 2H), 4.70 (s, 2H), 3.34 (m, 2H), 3.11 (m, 4H), 3.00 (t, 2H), 2.84 (t, 2H), 2.05 (q, 2H), 1.96 (m, 2H), 1.41 (m, 3H); ¹³C NMR (D₂O): δ 133.7, 133.1 (3C), 132.3 (2C), 130.7 (2C), 128.1 (2C), 125.0 (2C), 121.1 (2C), 52.1, 51.8, 47.3, 47.2, 39.3 (2C), 26.5, 23.5, 11.32. HRMS (FAB) calcd for C₂₃H₃₁N₃.3HCl (M+H-3HCl)⁺350.2591, Found 350.2588.

### EXAMPLE 2

### Synthesis of N-(4-Amino-butyl)-N′-anthracen-9-ylmethyl-N′-ethyl-butane-1,4-diamine Hydrochloride salt (7b)

A solution of the respective Boc-protected precursor (similar to molecule 11 but having a 4,4-triamine sequence; 140 mg, 0.26 mmole) was dissolved in absolute ethanol (2.28 mL) and stirred at 0° C. for 10 minutes. A 4N HCl solution (3.64 mL) was added to the reaction mixture dropwise and stirred at 0° C. for 20 minutes and then at room temperature overnight. The solution was concentrated in vacuo to give 7b as a yellow solid in 93% yield; ¹H NMR (D₂O) δ 8.76 (s, 1H), 8.23 (m, 4H), 7.78 (m, 2H), 7.68 (m, 2H), 5.33 (s, 2H), 3.43 (m, 2H), 3.19 (m, 2H), 3.04 (t, 2H), 3.00 (t, 2H), 2.83 (t, 2H), 1.74 (m, 6H), 1.47 (m, 5H); ¹³C NMR (CD₃OD): δ 133.0, 132.9, 132.6, 130.9, 129.4, 126.7, 124.3, 121.4, 53.4, 51.0, 40.1, 25.8, 24.7, 24.5, 22.3, 9.7. HRMS (FAB) calcd for C₂₅H₃₅N₃ (M-Cl)⁺: 377.2831, Found 377.2831.

### EXAMPLE 3

### Synthesis of {3-[(Anthracen-9-ylmethyl)-amino]-propyl}-(3-tert-butoxycarbonylamino-propyl)-carbamic acid tert-butyl ester (10)

To a stirred solution of amine 9 (1 g, 3.02 mmol) in 25% MeOH/CH₂Cl₂ (20 mL), was added a solution of 9-anthraldehyde 8 (0.519 g, 2.52 mmol) in 25% MeOH/CH₂Cl₂ (15 mL) under N₂. The mixture was stirred at room temperature overnight until the imine formation was complete (monitored by NMR). The solvent was removed in vacuo, the solid residue dissolved in 50% MeOH/CH₂Cl₂ (40 mL) and the solution cooled to 0° C. NaBH₄ (7.55 mmol) was added in small portions to the solution and the mixture was stirred at rt overnight. The solvent was removed in vacuo, the solid residue dissolved in CH₂Cl₂ (40 mL) and washed with Na₂CO₃ solution (10% aq. 3×30 mL). The CH₂Cl₂ layer was dried over anhydrous Na₂SO₄, filtered and removed in vacuo to give an oily residue. The oil was purified by flash column chromatography (5% MeOH/CHCl₃) to yield the product 10 as a pale yellow thick oil (0.38 g, 75%), R_{f}=0.3 (5% MeOH/CHCl₃); ¹H NMR (CDCl₃) δ 8.39 (s, 1H), 8.34 (d, 2H), 7.99 (d, 2H), 7.53 (m, 2H), 7.46 (m, 2H), 4.70 (s, 2H), 3.18-3.24 (m, 4H), 3.06 (br t, 2H), 2.85 (br t, 2H), 1.77 (br q, 2H), 1.60 (br q, 2H), 1.44 (m, 18H); ¹³C NMR (CDCl₃) δ 156.1 (2C), 131.6, 130.3 (2C), 129.2 (2C), 127.2 (2C), 126.1 (2C), 125.0 (3C), 124.2 (2C), 79.7 (2C), 53.7, 48.0, 46.0, 45.3, 44.0, 37.6, 29.6, 28.7 (6C), 27.5. HRMS (FAB) m/z calcd. for C₃₁H₄₃N₃O₄ (M+H)⁺ 522.3326, found 522.3304.

### EXAMPLE 4

### Synthesis of [3-(Anthracen-9-ylmethyl-ethyl-amino)-propyl]-(3-tert-butoxycarbonylamino-propyl)-carbamic acid tert-butyl ester (11)

Ethylbromide (EtBr, 508 mg, 4.66 mmol) was dissolved in anhydrous acetonitrile and added to a stirring mixture of compound 10 (805 mg, 1.55 mmol) and anhydrous K₂CO₃ (644 mg, 4.66 mmol). The mixture was then stirred overnight at 75° C. under a N₂ atmosphere. After the confirmation of the disappearance of the 10 by TLC, the solution was concentrated under reduced pressure. The residue was dissolved in CH₂Cl₂ (20 mL) and washed three times with aqueous sodium carbonate. The organic layer was separated, dried with anhydrous Na₂SO₄, filtered and concentrated under vacuum. Flash column chromatography of the residue gave 11 as a light yellow oil. Yield 80%; R_{f}=0.35 (3% MeOH/CHCl₃); ¹H NMR (CDCl₃) δ 8.43 (d, 2H), 8.25 (s, 1H), 7.86 (d, 2H), 7.43 (m, 2H), 7.37 (m, 2H), 4.34 (s, 2H), 2.78-2.92 (m, 4H), 2.64 (m, 4H), 2.36 (m, 2H), 1.21-1.48 (m, 23H), 1.16 (m, 2H); ¹³C NMR (CDCl₃) δ 155.7, 131.1 (3C), 131.0, 128.7 (2C), 127.0 (2C), 125.2 (2C), 124.8 (2C), 124.5 (2C), 78.9, 78.4, 53.4, 50.6, 50.0, 47.9, 45.3, 43.5, 37.1, 28.3 (6C), 26.5, 11.7. HRMS (FAB) m/z calcd. for C₃₃H₄₇N₃O₄ (M+H)⁺ 550.3639, found 550.3619.

### EXAMPLE 5

### Synthesis of Methanesulfonic acid 3-(anthracen-9-ylmethyl-ethyl-amino)-propyl ester (17)

To a solution of the alcohol 14b (216 mg, 0.74 mmol) and triethylamine (0.31 mL, 2.21 mmol) in CH₂Cl₂ (40 mL) at 0° C., methanesulfonyl chloride (253 mg, 2.21 mmol) was added dropwise over 30 minutes under a N₂ atmosphere. The reaction was stirred at 0° C. for 1 hour and slowly warmed to room temperature and stirred overnight under N₂. The reaction was then cooled to 0° C. and a 4M NaOH solution (20 mL) was added slowly with vigorous stirring. The organic phase was separated and washed with water (2×40 mL). The organic phase was separated and dried over anhydrous Na₂SO₄, filtered and concentrated to give the product 17 as a clear oil (94%) that was used in the next step without further purification. 17: R_{f}=0.54 (1% MeOH/CH₂Cl₂); ¹H NMR (CDCl₃) δ 8.47 (d, 2H), 8.36 (s, 1H), 7.98 (d, 2H), 7.52 (m, 2H), 7.47 (m, 2H), 4.43 (s, 2H, CH₂), 3.82 (t, 2H, OCH₂), 3.11 (s, 3H, CH₃), 2.70 (q, 2H, NCH₂), 2.51 (t, 2H, NCH₂), 1.66 (q, 2H, CH₂), 1.20 (t, 3H, CH₃).

### EXAMPLE 6

### Synthesis 3-[3-(Anthracen-9-ylmethyl-ethyl-amino)-propylamino]-propan-1-ol (18)

The mesylate 17 (384 mg, 1.04 mmol) and 3-amino-propanol (392 mg, 5.25 mmol) were dissolved in acetonitrile (20 mL). The mixture was then stirred at 75° C. under a N₂ atmosphere overnight. After the confirmation of the disappearance of the mesylate by TLC, the solution was concentrated under reduced pressure. The residue was dissolved in CH₂Cl₂ (20 mL) and washed three times with aqueous sodium carbonate. The organic layer was separated, dried with anhydrous Na₂SO₄, filtered and concentrated under vacuum. Flash column chromatography of the residue gave 18 as a light yellow oil. Yield 65%; R_{f}=0.35 (1:6:83 NH₄OH:MeOH:CH₂Cl₂); ¹H NMR (CDCl₃) δ 8.32 (d, 2H), 8.23 (s, 1H), 7.83 (d, 2H), 7.37 (m, 2H), 7.30 (m, 2H), 4.28 (s, 2H), 3.48 (t, 2H), 2.56 (q, 2H), 2.30 (t, 2H), 2.12 (t, 2H), 2.06 (t, 2H), 1.36 (q, 2H), 1.21 (q, 2H), 1.09 (t, 3H); ¹³C NMR (CDCl₃) δ 131.3, 131.2, 130.5, 129.3, 129.0, 127.3, 125.5, 125.3, 124.8, 124.8, 63.8, 50.8, 50.5, 49.5, 48.3, 47.7, 30.5, 26.6, 11.7. HRMS (FAB) m/z calcd. for C₂₃H₃₀N₂O (M+H)⁺ 351.2431; found 351.2430.

### EXAMPLE 6

### Synthesis of N-Anthracen-9-ylmethyl-N′-(3-ethoxy-propyl)-N-ethyl-propane-1,3-diamine (19)

The mesylate 17 (584 mg, 1.57 mmol) and 3-ethoxypropylamine (649 mg, 6.30 mmol) were dissolved in acetonitrile (30 mL). The mixture was then stirred at 75° C. under a N₂ atmosphere overnight. After the confirmation of the disappearance of the mesylate by TLC, the solution was concentrated under reduced pressure. The residue was dissolved in CH₂Cl₂ (25 mL) and washed three times with aqueous sodium carbonate. The organic layer was separated, dried with anhydrous Na₂SO₄, filtered and concentrated under vacuum. Flash column chromatography of the residue gave 19 as a light yellow oil. Yield 60%; R_{f}=0.35 (0.5:4:85.5 NH₄OH:MeOH:CH₂Cl₂); ¹H NMR (CDCl₃) δ 8.43 (d, 2H), 8.29 (s, 1H), 7.89 (d, 2H), 7.40 (m, 4H), 4.38 (s, 2H), 3.35 (q, 2H), 3.25 (t, 2H), 2.62 (q, 2H), 2.44 (t, 2H), 2.27 (t, 2H), 2.23 (t, 2H), 1.54 (q, 2H), 1.41 (q, 2H), 1.14 (m, 6H); ¹³C NMR (CDCl₃) δ 131.3, 131.2, 130.6, 128.9, 127.2, 125.4, 125.0, 124.7, 69.1, 66.0, 50.7, 50.6, 48.2, 47.5, 47.2, 30.1, 27.1, 15.4, 11.9.

### EXAMPLE 7

### Synthesis of N-Anthracen-9-ylmethyl-N′-(3-ethoxy-propyl)-N-ethyl-propane-1,3-diamine, Hydrochloride salt (20)

A solution of compound 18 (200 mg, 0.57 mmole) was dissolved in absolute ethanol (13 mL) and stirred at 0° C. for 10 minutes. A 4N HCl solution (22 mL) was added to the reaction mixture dropwise and stirred at 0° C. for 20 minutes and then at room temperature overnight. The solution was concentrated in vacuo to give 20 as a yellow solid in 95% yield. ¹H NMR (D₂O) δ 8.50 (s, 1H), 8.06 (m, 4H), 7.73 (m, 2H), 7.62 (m, 2H), 4.98 (s, 2H), 3.64 (t, 2H), 3.39 (q, 2H), 3.13 (t, 2H), 2.90 (t, 2H), 2.76 (t, 2H), 1.79 (q, 2H), 1.44 (m, 5H); ¹³C NMR (D₂O): δ 133.8, 133.4, 132.4, 130.8, 128.3, 125.1, 121.8, 61.4, 52.4, 52.1, 52.0, 48.0, 47.0, 30.5, 23.4, 11.3. HRMS (FAB) calcd for C₂₃H₃₀N₂O.2HCl (M+H-2HCl)⁺ 351.2431, Found 351.2428.

### EXAMPLE 8

### Synthesis of N-Anthracen-9-ylmethyl-N′-(3-ethoxy-propyl)-N-ethyl-propane-1,3-diamine, Hydrochloride salt (21)

A solution of compound 19 (169 mg, 0.45 mmole) was dissolved in absolute ethanol (13 mL) and stirred at 0° C. for 10 minutes. A 4NHC1 solution (22 mL) was added to the reaction mixture dropwise and stirred at 0° C. for 20 minutes and then at room temperature overnight. The solution was concentrated in vacuo to give 21 as a yellow solid in 95% yield. ¹H NMR (D₂O) δ 8.58 (s, 11H), 8.10 (m, 4H), 7.73 (m, 2H), 7.62 (m, 2H), 5.12 (s, 2H), 3.52 (m, 4H), 3.43 (q, 2H), 3.15 (t, 2H), 2.84 (t, 2H), 2.73 (t, 2H), 1.79 (m, 4H), 1.47 (t, 3H), 1.15 (t, 3H); ¹³C NMR (D₂O): δ 133.9, 133.5, 132.5, 130.9, 128.4, 125.2, 122.1, 69.9, 69.3, 52.7, 52.4, 52.2, 48.1, 47.0, 28.2, 23.5, 17.0, 11.4. HRMS (FAB) calcd for C₂₅H₃₄N₂O.2HCl (M+H-2HCl)⁺ 379.2749, Found 379.2749.

### EXAMPLE 9

### Synthesis of N-{3-[3-(Anthracen-9-ylmethyl-ethyl-amino)-propylamino]-propyl}-acetamide (23)

A saturated sodium carbonate solution (20 mL) was added to a vigorously stirred solution of 7a (250 mg, 0.55 mmol) in CH₂Cl₂ (20 mL). The organic layer was separated and was washed twice with saturated sodium carbonate. The combined organic layers were dried with anhydrous Na₂SO₄, filtered and concentrated to give the free amine 22 as a pale yellow oil in 99% yield. 22: ¹H NMR (CDCl₃) δ 8.48 (d, 2H), 8.38 (s, 1H), 7.97 (d, 2H), 7.47 (m, 4H), 4.47 (s, 2H), 2.70 (q, 2H), 2.51 (m, 4H), 2.29 (t, 2H), 2.17 (t, 2H), 1.56 (q, 2H), 1.24 (m, 5H); ¹³C NMR (CDCl₃): δ 131.5, 131.4, 130.9, 129.1, 127.4, 125.6, 125.5, 125.2, 124.9, 50.8, 50.7, 48.3, 47.8, 40.7, 33.7, 27.0, 12.0.

A mixture of compound 22 (190 mg, 0.54 mmol) and anhydrous K₂CO₃ (113 mg, 0.82 mmol) in anhydrous CH₂Cl₂ was stirred at 0° C. for 10 minutes. N-Acetoxysuccinimide (NHS ester, 60 mg, 0.38 mmol) was dissolved in dry CH₂Cl₂ and was added slowly to the above stirred solution at 0° C. under N₂ atmosphere. The mixture was then stirred for 30 minutes at 0° C. and then slowly allowed to come at room temperature and stirred for 8 hrs. After the confirmation of the disappearance of the ester by TLC in CH₂Cl₂/hexane (7:3), the solution was filtered and concentrated under reduced pressure. The residue was dissolved in CH₂Cl₂ (20 mL) and washed three times with aqueous sodium carbonate. The organic layer was separated, dried with anhydrous Na₂SO₄, filtered and concentrated under vacuum. Flash column chromatography of the residue gave 23 as a light yellow oil. Yield 67%; R_{f}=0.30 (7% MeOH: 1% NH₄OH1: 82% CH₂Cl₂); ¹H NMR (CDCl₃) δ 8.43 (d, 2H), 8.35 (s, 1H), 7.95 (d, 2H), 7.23 (m, 4H), 6.84 (br t, 1H), 4.43 (s, 2H), 3.05 (q, 2H), 2.71 (q, 2H), 2.43 (t, 2H), 2.20 (t, 2H), 2.09 (t, 2H), 2.78 (s, 3H), 2.48 (q, 2H), 1.21 (m, 5H); ¹³C NMR (CDCl₃) δ 170.0, 131.4, 131.3, 130.6, 129.1, 127.5, 125.7, 124.9, 124.9, 50.6, 48.1, 48.0, 48.0, 39.0, 28.2, 26.6, 23.4, 11.8. HRMS (FAB) m/z calcd. for C₂₅H₃₃N₃O (M+H)⁺ 392.2701, found 392.2709.

### EXAMPLE 10

### Synthesis of N-{3-[3-(Anthracen-9-ylmethyl-ethyl-amino)-propylamino]-propyl}-acetamide, Hydrochloride salt (24)

A solution of compound 23 (100 mg, 0.26 mmole) was dissolved in absolute ethanol (13 mL) and stirred at 0° C. for 10 minutes. A 4N HCl solution (22 mL) was added to the reaction mixture dropwise and stirred at 0° C. for 20 minutes and then at room temperature overnight. The solution was concentrated in vacuo to give 24 as a yellow solid in 96% yield. ¹H NMR (D₂O) δ 8.59 (s, 1H), 8.10 (m, 4H), 7.70 (m, 2H), 7.61 (m, 2H), 5.13 (s, 2H), 3.41 (br m, 2H), 3.12 (br m, 4H), 2.79 (br m, 4H), 1.98 (s, 3H), 1.71 (q, 2H), 1.43 (m, 5H); ¹³C NMR (D₂O): δ 174.5, 131.3, 130.8, 129.8, 128.2, 125.7, 122.5, 119.1, 50.0, 49.7, 49.4, 44.9, 44.1, 36.0, 25.5, 21.9, 20.8, 8.7. HRMS (FAB) calcd for C₂₅H₃₃N₃O.2HCl (M+H-2HCl)⁺ 392.2701, Found 392.2702.

### EXAMPLE 11

### Synthesis of {3-[(Anthracen-9-ylmethyl)-amino]-propyl}-carbamic acid tert-butyl ester (12a)

To a stirred solution of mono-BOC protected 1,3-diamine 15b (1 g, 5.75 mmol) in 25% MeOH/CH₂Cl₂ (20 mL), was added a solution of 9-anthraldehyde 8 (0.99 g, 4.8 mmol) in 25% MeOH/CH₂Cl₂ (15 mL) under N₂. The mixture was stirred at room temperature overnight until the imine formation was complete (monitored by NMR). The solvent was removed in vacuo, the solid residue dissolved in 50% MeOH/CH₂Cl₂ (40 mL) and the solution cooled to 0° C. NaBH₄ (14.42 mmol) was added in small portions to the solution and the mixture was stirred at rt overnight. The solvent was removed in vacuo, the solid residue dissolved in CH₂Cl₂ (40 mL) and washed with Na₂CO₃ solution (10% aq. 3×30 mL). The CH₂Cl₂ layer was dried over anhydrous Na₂SO₄, filtered and removed in vacuo to give an oily residue. The oil was purified by flash column chromatography (5% MeOH/CHCl₃) to yield the product 12a as a pale-yellow, thick oil (75%). R_{f}=0.3 (5% MeOH/CHCl₃); ¹H NMR (CDCl₃) δ 8.20 (m, 3H), 7.85 (d, 2H), 7.43 (m, 2H), 7.36 (m, 2H), 5.32 (t, 1H), 4.52 (s, 2H), 3.10 (q, 2H), 2.77 (t, 2H), 1.56 (q, 2H), 1.39 (s, 9H); ¹³C NMR (CDCl₃) δ 156.0, 131.3 (2C), 130.1 (2C), 129.0 (2C), 127.0 (2C), 125.9 (2C), 124.8 (2C), 124.0 (2C), 78.7 (2C), 48.5, 45.8, 39.4, 29.9, 28.5. HRMS (FAB) m/z calcd. for C₂₃H₂₈N₂O₂ (M+H)⁺ 365.2224; found 365.2208.

### EXAMPLE 12

### Synthesis of [3-(Anthracen-9-ylmethyl-ethyl-amino)-propyl]-carbamic acid tert-butyl ester (12b)

Bromoethane (489 mg, 4.48 mmol) was dissolved in anhydrous acetonitrile and was added to the stirring mixture of compound 12a (545 mg, 1.5 mmol) and anhydrous K₂CO₃ (620 mg, 4.48 mmol). The mixture was then stirred at 75° C. under a N₂ atmosphere overnight. After the confirmation of the disappearance of the 12a by TLC, the solution was concentrated under reduced pressure. The residue was dissolved in CH₂Cl₂ (20 mL) and washed three times with aqueous sodium carbonate. The organic layer was separated, dried with anhydrous Na₂SO₄, filtered and concentrated under vacuum. Flash column chromatography of the residue gave 12b as a light yellow oil. Yield 80%; R_{f}=0.35 (3% MeOH/CHCl₃); ¹H NMR (CDCl₃) δ 8.39 (d, 2H), 8.30 (s, 1H), 7.90 (d, 2H), 7.40 (m, 4H), 4.54 (br t, 1H), 4.37 (s, 2H), 2.74 (q, 2H), 2.60 (q, 2H), 2.39 (t, 2H), 1.44 (q, 2H), 1.31 (m, 9H), 1.12 (t, 3H); ¹³C NMR (CDCl₃) δ 155.7, 131.3 (2C), 131.2 (2C), 130.4 (2C), 129.0 (2C), 127.5 (2C), 125.7 (2C), 124.8 (2C), 78.3, 50.6, 50.5, 47.7, 39.1, 28.6 (3C), 26.6, 11.8. HRMS (FAB) m/z calcd. for C₂₅H₃₂N₂O₂ (M+H)⁺ 393.2537; found 393.2523.

### EXAMPLE 13

### Synthesis of N¹-Anthracen-9-ylmethyl-N¹-ethyl-propane-1,3-diamine, Hydrochloride salt (12c)

A solution of 12b (400 mg, 1.02 mmole) was dissolved in absolute ethanol (13 mL) and stirred at 0° C. for 10 minutes. A 4N HCl solution (22 mL) was added to the reaction mixture dropwise and stirred at 0° C. for 20 minutes and then at room temperature overnight. The solution was concentrated in vacuo to give 12c as a yellow solid in 90% yield. ¹H NMR (D₂O) δ 8.42 (s, 1H), 8.0 (m, 4H), 7.71 (m, 2H), 7.61 (m, 2H), 4.87 (s, 2H), 3.34 (br q, 2H), 3.14 (br t, 2H), 2.81 (t, 2H), 2.00 (q, 2H), 1.40 (t, 3H); ¹³C NMR (D₂O): δ 133.8, 133.3 (3C), 132.4 (2C), 130.7 (2C), 128.2 (2C), 125.1 (2C), 121.5 (2C), 52.0, 51.9, 39.2 (2C), 24.5, 11.3. HRMS (FAB) calcd for C₂₀H₂₄N₂.2HCl (M+H-2HCl)⁺ 293.2012, Found 293.2009.

### EXAMPLE 14

### Synthesis of 3-[(Anthracen-9-ylmethyl)-amino]-propan-1-ol, 14a

To a stirred solution of 3-amino-1-propanol, 15a (0.87 g, 11.65 mmol) in 25% MeOH/CH₂Cl₂ (20 mL), was added a solution of aldehyde 8 (2.00 g, 9.7 mmol) in 25% MeOH/CH₂Cl₂ (15 mL) under N₂. The mixture was stirred at room temperature overnight until the imine formation was complete (monitored by NMR). The solvent was removed in vacuo, the solid residue dissolved in 50% MeOH/CH₂Cl₂ (40 mL) and the solution was cooled to 0° C. NaBH₄ (29.1 mmol) was added in small portions to the solution and the mixture was stirred at rt overnight. The solvent was removed in vacuo, the solid residue dissolved in CH₂Cl₂ (40 mL) and washed with 10% aq. Na₂CO₃ solution (3×30 mL). The CH₂Cl₂ layer was separated, dried over anhydrous Na₂SO₄, filtered and removed in vacuo to give an oily residue. The oil was purified by flash column chromatography (6% MeOH/CHCl₃) to yield the product 14a as a pale yellow thick oil (78%), R_{f}=0.3 (6% MeOH/CHCl₃); ¹H NMR (CDCl₃) δ 8.39 (s, 1H), 8.27 (d, 2H), 7.99 (d, 2H), 7.52 (m, 2H), 7.45 (m, 2H), 4.71 (s, 2H), 3.79 (t, 2H), 3.09 (t, 2H), 1.74 (q, 2H).

### EXAMPLE 15

### Synthesis of 3-(Anthracen-9-ylmethyl-ethyl-amino)-propan-1-ol, 14b

Bromoethane (616 mg, 5.65 mmol) was dissolved in anhydrous acetonitrile and was added to the stirring mixture of compound 14a (500 mg, 1.9 mmol) and anhydrous K₂CO₃ (781 mg, 5.7 mmol). The mixture was then stirred overnight at 75° C. under a N₂ atmosphere. After confirmation of the disappearance of 14a by TLC, the solution was concentrated under reduced pressure. The residue was dissolved in CH₂Cl₂ (20 mL) and washed three times with aqueous sodium carbonate. The organic layer was separated, dried with anhydrous Na₂SO₄, filtered and concentrated under vacuum. Flash column chromatography of the residue gave 14b as a light yellow oil. Yield 82%; R_{f}=0.35 (3% MeOH/CHCl₃); ¹H NMR (CDCl₃) δ 8.28 (m, 3H), 7.86 (d, 2H), 7.44 (m, 2H), 7.36 (m, 2H), 4.29 (s, 2H), 3.15 (t, 2H), 2.65 (q, 2H), 2.52 (t, 2H), 1.43 (q, 2H), 1.16 (t, 3H); ¹³C NMR (CDCl₃) δ 131.2, 131.1, 129.3, 129.0, 127.6, 125.8, 124.8, 124.4, 63.4, 52.6, 50.5, 47.7, 28.1, 11.6. HRMS (FAB) m/z calcd. for C₂₀H₂₃NO (M+H)⁺ 294.1852; found 294.1859.

### EXAMPLE 16

### Synthesis of N1-Anthracen-9-ylmethyl-propane-1,3-diamine, Hydrochloride salt (16)

A solution of 12a (200 mg, 0.51 mmole) was dissolved in absolute ethanol (6 mL) and stirred at 0° C. for 10 minutes. A 4N HCl solution (10 mL) was added to the reaction mixture dropwise and stirred at 0° C. for 20 minutes and then at room temperature overnight. The solution was concentrated in vacuo to give 16 as a yellow solid in 90% yield. ¹H NMR (CD₃OD) δ 8.69 (s, 1H), 8.39 (d, 2H), 8.15 (d, 2H), 7.74 (m, 2H), 7.60 (m, 2H), 5.33 (s, 2H), 3.45 (t, 2H), 3.12 (t, 2H), 2.23 (q, 4H); ¹³C NMR (CD₃OD): δ 132.6, 132.0, 131.6, 130.6, 129.0, 126.7, 124.0, 122.5, 46.6, 44.6, 38.1, 25.4. HRMS (FAB) calcd for C₁₈H₂₀N₂.2HCl (M+H-2HCl)+265.1699, Found 265.1704.

### EXAMPLE 17

### Biological Evaluation of Polyamine Derivatives

In terms of Table 1, L1210 cells are mouse leukemia cells and are the gold standard in terms of evaluating polyamine cytotoxicity data due to a plethora of prior data in this cell line for other polyamine structures. Clearly 7a was similar in all respects to 4a (Table 1). The fact that similar alterations in the structure of 7a and 4a gave the same biological response suggests that they are hitting the same biological target. Low IC₅₀ values in Table 1 suggest greater cytotoxicity of the drug. The lower the Kᵢ value, the higher the affinity of the drug for the polyamine transporter (PAT) on the cell surface. Chinese hamster ovary (CHO) cells and a mutant line without an active PAT (CHO-MG) also evaluated the PAT selectivity of these drugs. High (CHO-MG/CHO) IC₅₀ ratios suggest a highly selective PAT substrate. Inspection of Table 1 suggests that 7a is an effective mimic of 4a (both have low IC₅₀ values), but does not use the PAT for cellular entry (both have low CHO-MG/CHO IC₅₀ ratio).

TABLE 1
Biological evaluation of polyamine derivatives in L1210, CHO and CHO-MG cells.^{a}

| | L1210 | L1210 | | CHO-MG | CHO | IC₅₀ |
|---|---|---|---|---|---|---|
| Compd (tether) | IC₅₀ in μM | Kᵢ value (μM) | Ref | IC₅₀ in μM | IC₅₀ in μM | Ratio^{b} |
| 4a: dihydroMotu (3,3) | 3.0 (±0.5) | 9.9 (±0.5) | 3 | 10.0 (±2.6) | 10.5 (±1.6) | 1 |
| 4b: dihydroMotu (4,4) | 18.5 (±2.9) | 6.2 (±0.5) | 3 | 28.2 (±5.6) | 30.0 (±4.1) | 1 |
| 6a: Ant-methyl (3,3) | 1.8 (±0.4) | 33.4 (±2.6) | 3 | 3.4 (±0.5) | 1.9 (±0.4) | 1.8 |
| 6b: Ant-methyl(4,4) | 0.30 (±0.04) | 1.8 (±0.1) | 3 | 66.7 (±4.1) | 0.45 (±0.10) | 148 |
| 6c: Ant-ethyl(4,4) | 3.5 (±0.7) | 1.6 (±0.1) | 8 | 33.5 (±7.1) | 9.8 (±1.1) | 3.4 |
| 6d: Ant-propyl(4,4) | 76.3 (±4.8) | 1.1 (±0.1) | 8 | 130.8 (±5.5) | 130.1 (±7.1) | 1 |
| 7a: N¹-ethyl-N¹-Ant- | 2.2 (±0.1) | 23.5 (±0.9) | | 4.0 (±0.3) | 5.3 (±0.4) | 0.8 |
| methyl (3,3) | | | | | | |
| 7b: N¹-ethyl-N¹-Ant- | 22.2 (±1.2) | 24.4 (±1.5) | 3 | 21.9 (±0.9) | 22.2 (±0.7) | 1 |
| methyl (4,4) | | | | | | |

^{a}Definitions used in Table 1, column 1: Ant = anthracen-9-yl, dihydroMotu = dihydromotuporamine; column 4: Ref denotes the reference number in which the data was originally reported. A blank in the Ref column denotes new data. Cells were incubated for 48 h with the respective conjugate.
^{b}The IC₅₀ ratio denotes the (CHO-MG/CHO) IC₅₀ ratio, a measure of PAT selectivity.

### EXAMPLE 18

### Inhibition of Motuporamine Mimic Agents

DihydroMotuporamine C, 4a, was used to develop an imaginal disc assay in Drosophila flies. The imaginal leg discs were collected by microscopic dissection from maggots. The assay reproducibly showed that (at 18 μM) 4a gave very high inhibition (≧87%) of development of the imaginal disc (Table 2). Inhibition was measured as failure of the disc to fully develop into a fly leg after 15 hr of incubation in Robb's growth medium. This presumably occurs by overactivation of Rho, an important signaling pathway in development. Hyper-stimulation of this pathway is sufficient to block development of the fly leg.

Using this concentration (18 μM) the panel of mimics were assayed (4, 6a, 6b, 7a, 7b, 12c, 13b, 16, 20, 21, and 24). As shown in Table 2, all of the derivatives gave medium to high levels of inhibition in the assay, except 4b and 7b. In this regard, most of the new materials provided similar inhibition as 4a.

Note: the disc assay itself is binary. The tested compound is either as good as or worse than 4a. Compounds that are better than 4a will only give the same maximal response (90-100% inhibition). Future work will be necessary to see which of the most active compounds (6a, 6b, 7a, 12c, 13b, 16, 20, 21, and 24) are best in vivo in terms of slowing the spread of cancers (anti-metastatic activity).

Fly Stocks: 20 female and 5 male wild type Oregon variant of Drosophila melanogaster interbred in blue food medium for 24 hours then flies are removed. Third instar wall-crawling larvae collected then dissected on the sixth day. Flies kept in 25° C. incubation chamber.

Blue Food Preparation: Standard corn meal medium heated then mixed completely with aqueous 1% Bromophenol Blue. The food medium is cooled for one day or more before use.

Dissection Medium: Ringer's Buffer with 10 uL 0.1% BSA. Ringer's Medium consists of 130 mM NaCl, 5 mM KCl, 1.5 mM CaCl₂-2H₂O, Stored at room temperature (23° C.), BSA added before dissection.

Cultivation Medium: Minimal Robb's Medium with 10 uL 0.1% BSA. Minimal Robb's Medium consists of 40 mM KCl, 0.4 mM KH₂PO₄, 40 mM NaCl, 0.4 mM NaH₂PO₄.7H₂O, 1.2 mM MgSO₄.7H₂O, 1.2 mM MgCl₂-6H₂O, 1 mM CaCl₂-2H₂O, 10 mM Glucose, 0.2 mM L-asparagine, 4.0 mM L-glutamine, 0.16 mM Glycine, 0.64 mM L-leucine, 0.32 mM L-proline, 0.16 mM R-Serine, 0.64 mM L-valine. Stored at −25° C. One day before dissection, aliquot defrosted in 4° C. Add BSA then warmed to room temperature (23° C.) before dissection.

0.1% BSA: 0.1 g BSA fraction V (Sigma#A-9647) in 10 mL distilled H₂O, Stored at 4° C.

Developmental Hormone: 1 mg 20-hydroxyecdysone (Sigma #H-5142) in 1 mL 100% Ethanol. Stored at −25° C. Before use, stock is diluted 10× (1 mL added to 9 mL of 100% Ethanol). 10 μL of diluted 20-hydroxyecdysone is added to culture.

Dissection Procedures: Third instar larvae are removed from cultivation bottle with a wetted brush and washed in dH₂O to remove food medium clinging to larvae. The cleaned larvae are dissected in Ringer's Medium using forceps. The imaginal discs are washed in fresh Ringer's Medium, and then cultivated with 1 mL of Robb's Medium in 12-well culture plate. Each culture well should have 30˜40 imaginal discs.

Culturing Procedures: In a larger container, place the 12-well culture plate on a moist towel, then seal the large container. Cultivate at 25° C. for 15 hours. Evaluation: Three categories will be used to grade the eversion of each imaginal disc. Full Eversion—the leg is fully extended from the disc. Partial Eversion—the leg is protruding from the epithelial. No Eversion—no sign of any protrusion.

TABLE 2
Eversion Inhibition by new compounds at
18 μM in an Imaginal Disc Assay^{a}

| Compound | % Inhibition |
|---|---|
| 4a (Motu 3,3) | 87 |
| 4b (Motu 4,4) | 0 |
| 6a (Ant 3,3) | 60 |
| 6b (Ant 4,4) | 97 |
| 7a (AntNEt 3,3) | 95 |
| 7b (AntNEt 4,4) | 8 |
| 12c (AntNEtDiamine 3) | 79 |
| 13b (Ant Diamine 4) | 91 |
| 16 (Ant Diamine 3) | 92 |
| 20 (Ant NEt aminoalcohol 3,3) | 38 |
| 21 (AntNEt aminoether 3,3) | 36 |
| 24 (AntNEt acetamide 3,3) | 58 |

^{a}The error is typically near 10-15% for this type of developmental measurement.

### EXAMPLE 19

### Synthesis and Characterization of N-Anthracen-9-ylmethyl-N′-(4-ethylamino-butyl)-butane-1,4-diamine, Hydrochloride salt and N-Anthracen-9-ylmethyl-N′-(4-methylamino-butyl)-butane-1,4-diamine, Hydrochloride salt

Compound 6b was used in the synthesis of the N⁹-ethyl derivative 27 (Wang et al., J. Med. Chem. 2003 46:2663-2671). The selective N-alkylation of the primary amine in 6b was accomplished with the cesium method reported by Jung (Salvatore et al., J. Org. Chem., 2002, 67:674-683). When CsOH.H2O (1 equiv) in DMF, 4 Å molecular sieves, and ethyl bromide were used, the secondary amine 25 was synthesized in 21% yield. Some of the N,N-diethylated compound was also separated by column chromatography. Further treatment of 25 with 4 N HCl resulted in compound 27. To confirm the structure of compounds 27 and 25, di-tert-butyldicarbonate was used in excess to Boc-protect all the available amines of 27 to synthesize the tricarbamate 26. The ¹H NMR spectrum of 26 showed the presence of three Boc groups and the absence of a doublet of triplets at 3.03 ppm, which indicated that a RCH2CH2NHBOC group was not present. This observation confirmed that there is no NH carbamate available in compound 26. Both observations confirmed the regiochemistry of the N-ethyl group in compounds 25 and 27. Indeed, a comparison of the 1H NMR spectra of 35, 36, and 27 showed distinct differences and ruled out conclusively any misassignment of the N-Et regiochemistry.

As shown in Scheme 3, the synthesis of compound 28 utilized the previously synthesized alcohol 29 (Kaur et al., J. Med. Chem., 2005 48:3832-3839). The HCl salt of amine 30 was obtained using 4 N HCl in ethanol. Regioselective reductive amination of amine 30 with 9-anthraldehyde in the presence of TEA resulted in the desired secondary amine 31 in95% yield. Reaction of 31 with di-tert-butyl dicarbonate provided the alcohol 32 in 96% yield. 5 Mesylation of alcohol 32 resulted in mesylate 33, which was reacted with excess methylamine to obtain amine 34 in 73% yield. Lastly, removal of the Boc groups with 4 N HCl provided the N9-methyl derivative 28 in 95% yield.

Biological Evaluation. Once synthesized, the conjugates were screened for cytotoxicity in L1210, CHO and CHO-MG cells. L1210 (mouse leukemia) cells were selected to enable comparisons with the published IC₅₀ and Kᵢ values for a variety of polyamine substrates. Chinese Hamster Ovary (CHO) cells were chosen along with a mutant PAT deficient cell line (CHO-MG) in order to comment on selective transport via the PAT (Wang et al, J. Med. Chem. 2003, 46, 2663-2671; Wang et al, J. Med. Chem. 2003, 46, 2672-2682; Wang et al., J. Med. Chem. 2003, 46, 5129-5138; Delcros et al., J. Med. Chem., 2002, 45, 5098-5111). The results are shown in Table 3.

TABLE 3
Biological Evaluation of polyamine derivatives in L1210,
CHO and CHO-MG cells in the presence of AG.^{a}

| | L1210 | L1210 Kᵢ | CHO-MG | CHO | IC₅₀ |
|---|---|---|---|---|---|
| Compd (tether) | IC₅₀ in μM | values (μM) | IC₅₀ in μM | IC₅₀ in μM | Ratio^{b} |
| 6b: Ant- | 0.30 (±0.04) | 1.80 (±0.10) | 66.7 (±4.1) | 0.45 (±0.10) | 148 |
| methyl(4,4) | | | | | |
| 27: N⁹-ethyl-N¹- | 1.01 (±0.04) | 3.5 (±0.2) | 57.9 (±2.3) | 9.82 (±0.28) | 5.9 |
| Antmethyl (4,4) | | | | | |
| 28: N⁹-methyl- | 0.40 (±0.05) | 8.2 (±0.6) | 60.0 (±2.6) | 4.88 (±0.15) | 12.3 |
| N¹-Antmethyl | | | | | |
| (4,4) | | | | | |

^{a}Definitions used in Table 1, column 1: Ant = anthracen-9-yl, column 4. Cells were incubated for 48 h at 37° C. with the respective conjugate.
^{b}The ratio denotes the (CHO-MG/CHO) IC₅₀ ratio, a measure of PAT selectivity.

L1210 IC₅₀ and Kᵢ studies. The IC₅₀ values listed in Table 3 represent the concentration of the polyamine conjugate required to reduce the relative cell growth by 50%. The Kᵢ values in Table 3 were determined for [¹⁴C]spermidine uptake and reflect the affinity of the polyamine derivative for the polyamine transporter on the cell surface. With both parameters, one can determine whether high affinity for the transporter (e.g., low Kᵢ value) translated into high cytotoxicity (e.g., low IC₅₀ value), etc.

Although the Kᵢ values provide relative affinity measures of polyamine derivatives towards the PAT system, it has been previously shown that they did not always correlate with the observed cytotoxicity. This is likely due to the fact that polyamine transport is a multi-step process, which involves cell surface interactions followed by uptake across the cell membrane. In addition, some polyamine derivatives have been shown to have a high affinity for the PAT, but are not transported into the cells.

In the L1210 experiments, the higher Kᵢ value of 34 correlated with its lower cytotoxicity, IC₅₀ of 34: 22.2 μM. Indeed, the IC₅₀ value increased significantly from the parent system, 6b (0.3 μM). This revealed that at least for these compounds both the PAT binding affinity and the conjugate's cytotoxicity were sensitive to the degree of alkylation at the NM position. As the position of the tertiary amine was moved from N¹ (35) to N⁵ (36) and N⁹ (27), the cytotoxicity increased (IC₅₀ value: 35: 22.2 μM; 36: 0.88 μM; 27: 1.01 μM). In terms of N⁹-substituent effects, the small N⁹-alkyl groups of 27 and 28 did not significantly alter the cytotoxicity profile seen with the parent 6b (e.g., IC₅₀ 6b: 0.30 μM; 27: 1.01 μM; IC₅₀ 28: 0.40 EM). Indeed, a spermidine rescue experiment, wherein the competitive antagonist spermidine is added, showed significant rescue of cells from compound 28. Specifically, a significant increase in the IC₅₀ value was observed in the presence of added SPD (IC₅₀ 28: 0.4 μM; IC₅₀ 28+SPD; 1.38 μM). This result suggests that 28 is able to access cells via the same polyamine transporter in L1210 cells as utilized by the native spermidine. In general, the spermidine (SPD) protection assays were performed to determine whether uptake of selected polyamine analogues is mediated, in part or in whole, by the polyamine transport apparatus (PAT). To answer this question, competition assays were performed in the absence and presence of SPD. To maximize the protective effects of spermidine (SPD), an excess of SPD (200 μM) was used during these experiments. Use of an excess of SPD ensures that SPD is transported into the cell and, hence, provides a high level of competition with the selected polyamine derivatives for the PAT protein. Note: a similar SPD protection effect was observed in CHO cells for compounds 6b, 27, and 28.

CHO and CHO-MG studies. Chinese hamster ovary (CHO) cells were chosen along with a mutant cell line (CHO-MG) in order to comment on how the synthetic conjugates gain access to cells. The CHO-MG cell line is polyamine-transport deficient and was isolated after selection for growth resistance to methylglyoxalbis(guanylhydrazone), MGBG, (CH₃C[═N—NHC(═NH)NH₂]CH[═N—NHC(═NH)NH₂]) using a single-step selection after mutagenesis with ethylmethanesulfonate (Mandel et al., J. Cell. Physiol., 1978, 97, 335-344; Byers et al., Biochem. J. 1989, 263, 745-752).

For the purposes of this study, the CHO-MG cell line represents cells with no PAT activity and provided a model for alternative modes of entry or action, which are independent of PAT. These alternative modes of entry include passive diffusion or utilization of another transporter. The alternative modes of action may also include interactions on the outer surface of the plasma membrane or other membrane receptor interactions.

In contrast, the parent CHO cell line represents a cell type with high PAT activity. Comparison of conjugate cytotoxicity in these two CHO lines provided an important screen to detect selective conjugate delivery via the PAT. For example, a conjugate with high utilization of the polyamine transporter would be very toxic to CHO cells, but less so to CHO-MG cells. In short, highly-selective PAT ligands should give high (CHO-MG/CHO) IC₅₀ ratios.

Dramatic differences in cytotoxicity were observed with 6b (CHOMG/CHO IC₅₀ ratio: 148), a highly PAT-selective substrate. The CHOMG/CHO IC₅₀ ratios listed in Table 3 suggested that PAT targeting is influenced by the degree of substitution of nitrogen at the N¹ position of the polyamine vector. A direct correlation was observed between cytotoxicity and polyamine conjugate uptake. Therefore, the relative toxicities observed in CHO and CHOMG cells represent a measure of differential uptake via PAT and provide a measure of PAT selectivity.

The CHOMG/CHO IC₅₀ ratios revealed that PAT selectivity was very sensitive to alkylation at N¹ and N⁵ with CHOMG/CHO ratios of 1 and 1.8, respectively. Interestingly, N⁹-ethylation of 6b provided compound 27, which was 5.9 times more toxic to CHO than CHOMG cells. Although compound 6b was more selective in using PAT, the N⁹ ethyl amine derivative 27, could also be accommodated by PAT, albeit to a lesser degree of selectivity. However, previous results showed that larger N⁹-substituents resulted in the complete loss of PAT selectivity. Pursuing this insight, compound 28 with a methyl group at the N⁹ position was evaluated in the CHO cell lines. The smaller N⁹ methyl substituent resulted in increased PAT selectivity. The increased PAT selectivity of N-methyl analogue 28 over its N-ethyl counterpart 27 is likely due to steric effects wherein, the smaller substituent at the N⁹ position is better accommodated by PAT. Indeed, the ability to target PAT increased as one reduced the size of the N⁹ substituent within the series: 27, 28, and 6b.

N-Alkylated polyamines have been shown to have enhanced metabolic stability due to their ability to avoid degradation by serum amine oxidases (present in the culture medium) and by the intracellular polyamine oxidase (PAO). Aminoguanidine (AG) is a known inhibitor of the serum amine oxidases and is routinely added (at 2 mM) during our cell culture experiments to avoid polyamine drug degradation by the serum oxidase. It was speculated that in the absence of AG, the polyamine conjugates could be degraded by the serum oxidases and converted to other metabolites, which could affect the measured cytotoxicity and PAT selectivity of the conjugates. Prior experiments revealed that polyamine metabolic stability could be modulated via steric effects. Therefore, even though the N⁹-alkylated polyamine conjugates 27 and 28 showed lower PAT selectivity than the lead compound 6b, it was possible that they were more metabolically stable. To test this hypothesis, we determined the cytotoxicity of 6b, 27 and 28 in the presence and absence of AG (Table 4).

TABLE 4
Biological Evaluation of polyamine derivatives in CHO and
CHO-MG cells in the absence of AG (IC₅₀ values in μM).^{a}

| | CHO-MG | CHO | IC₅₀ | IC₅₀ |
|---|---|---|---|---|
| | IC₅₀ | IC₅₀ | Ratio^{b} | Ratio^{b} |
| Compd (tether) | w/o AG | w/o AG | w/o AG | with AG |
| 6b: Antmethyl | 7.05 (±0.31) | 1.74 (±0.07) | 4 | 148 |
| (4,4) | | | | |
| 7: N⁹-ethyl- | 51.21 (±1.81) | 13.17 (±0.45) | 4 | 5.9 |
| N¹-Antmethyl | | | | |
| (4,4) | | | | |
| 8: N⁹-methyl- | 56.21 (±1.95) | 4.89 (±0.13) | 11.5 | 12.3 |
| N¹-Antmethyl | | | | |
| (4,4) | | | | |

^{a}Definitions used in Table, column 1: Ant = anthracen-9-yl, Cells were incubated for 48 h with the respective conjugate;
^{b}the ratio denotes the (CHO-MG/CHO) IC₅₀ ratio, a measure of PAT selectivity.

The PAT selectivity of lead compound 6b was lowered from 148 to 4 (Table 4) in the absence of AG, which clearly suggests that 6b is a substrate for serum amine oxidases. In contrast, the PAT selectivities of N⁹-alkylated compounds 27 and 28 were retained in the absence of AG suggesting that these compounds are not the substrates for serum amine oxidases and are still able to utilize the polyamine transporter. In the absence of AG, compounds 27 and 6b have the same PAT selectivity (ratio IC₅₀ CHOMG/CHO: 4). However, in the absence of AG the N⁹-methyl analogue 28 has a higher selectivity (ratio IC₅₀ ratio: 11.5) than either its N-ethyl derivative 27 or the parent system, 6b.

### EXPERIMENTAL

Materials. Silica gel (32-63 μm) and chemical reagents were purchased from commercial sources and used without further purification. All solvents were distilled prior to use. ¹H and ¹³C NMR spectra were recorded at 300 and 75 MHz, respectively. TLC solvent systems are based on volume % and NH₄OH refers to concentrated aqueous NH₄OH. Elemental analyses were performed by Atlantic Microlabs (Norcross, Ga.).

Biological studies. Murine leukemia cells (L1210), CHO and CHO-MG cells were grown in RPMI medium supplemented with 10% fetal calf serum, glutamine (2 mM), penicillin (100 U/mL), streptomycin (50 μg/mL). L-Proline (2 μg/mL) was added to the culture medium for CHO-MG cells. Cells were grown at 37° C. under a humidified 5% CO₂ atmosphere. Aminoguanidine (AG, 2 mM) was added to the culture medium to prevent oxidation of the drugs by the enzyme (bovine serum amine oxidase) present in calf serum. Trypan blue staining was used to determine cell viability before the initiation of a cytotoxicity experiment. Cells in early to mid log-phase were used. IC₅₀ determinations. Cell growth was assayed in sterile 96-well microtiter plates (Becton-Dickinson, Oxnard, Calif., USA). L1210 cells were seeded at 5e⁴ cells/mL of medium (100 μL/well). CHO and CHO-MG cells were plated at 2e³ cells/mL. Drug solutions (10 μL per well) of appropriate concentration were added at the time of seeding for L1210 cells and after an overnight incubation for the CHO cell lines. After exposure to the drug for 48 hr, cell growth was determined by measuring formazan formation from 3-(4,5-dimethylthiazol-2-yl)2,5-diphenyltetrazolium using a Titertek Multiskan MCC/340 microplate reader for absorbance (540 nm) measurements (Mosmann, T., J. Immunol. Methods 1983, 65, 55-63).

Kᵢ procedure. The ability of the conjugates to interact with the polyamine transport system was determined by measuring competition by the conjugates against radiolabeled spermidine uptake in L1210 cells. This procedure was used to obtain the data listed in Table 3. Initially, the Kₘ value of spermidine transport was determined as previously described (Clément et al., Biochem. J. 1995, 312, 933-938). The ability of conjugates to compete for [¹⁴C]spermidine uptake were determined in L1210 cells by a 10-min uptake assay in the presence of increasing concentrations of competitor, using 1 μM [¹⁴C]spermidine as substrate. Kᵢ values for inhibition of spermidine uptake were determined using the Cheng-Prusoff equation (Cheng and Prusoff, Biochem. Pharmacol. 1973, 22, 3099-3108) from the IC₅₀ value derived by iterative curve fitting of the sigmoidal equation describing the velocity of spermidine uptake in the presence of the respective competitor (Torossian et al., Biochem. J., 1996, 319, 21-26). L1210 cells were grown and maintained according to established procedures (Bergeron et al., J. Med. Chem. 2000, 43, 224-235) and were washed twice in HBSS prior to the transport assay.

N-Anthracen-9-ylmethyl-N′-(4-ethylamino-butyl)-butane-1,4-diamine, Hydrochloride salt, 27: A solution of 25 (49 mg, 0.13 mmol) was dissolved in absolute ethanol (6 mL) and stirred at 0° C. for 10 minutes. A 4N HCl solution (11 mL) was added to the reaction mixture dropwise and stirred at 0° C. for 20 minutes and then at room temperature overnight. The solution was concentrated in vacuo to give 27 as a yellow solid in 93% yield. ¹H NMR (D₂O) δ 8.64 (s, 1H), 8.22 (d, 2H), 8.13 (d, 2H), 7.71 (m, 2H), 7.61 (m, 2H), 5.19 (s, 2H), 3.28 (t, 2H), 3.09 (m, 8H), 1.77 (m, 8H), 1.30 (t, 3H); ¹³C NMR (D₂O): δ 133.7, 133.3, 133.1, 132.2, 130.5, 128.3, 125.3, 123.5, 49.9, 49.6, 49.1, 45.7, 25.7, 13.4, HRMS (FAB) calcd for C₂₅H₃₅N₃.3HCl(M+H-3HCl)⁺ 378.2909, Found 378.2906.

N-Anthracen-9-ylmethyl-N′-(4-methylamino-butyl)-butane-1,4-diaine, Hydrochloride salt, 28: A solution of BOC-protected 34 (180 mg, 0.32 mmole) was dissolved in absolute ethanol (13 mL) and stirred at 0° C. for 10 minutes. A 4N HCl solution (22 mL) was added to the reaction mixture dropwise and stirred at 0° C. for 20 minutes and then at room temperature overnight. The solution was concentrated in vacuo to give 28 as a yellow solid in 95% yield. ¹H NMR (300 MHz, D₂O) δ 8.32 (s, 1H), 7.99 (d, 2H), 7.93 (d, 2H), 7.60 (m, 2H), 7.50 (m, 2H), 4.87 (s, 2H), 3.16 (t, 2H), 3.02 (m, 6H), 2.70 (s, 3H), 1.72 (m, 8H); ¹³C NMR (D₂O): δ 130.7, 130.4, 130.1, 129.5, 127.7, 125.5, 122.5, 120.4, 48.4, 47.2, 47.1, 47.0, 42.8, 32.9, 23.1, 23.0, 22.9. HRMS (FAB) calcd for C₂₄H₃₃N₃.3HCl (M+H-3HCl) 364.2747 Found; 364.2715.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims. The teachings of all references cited herein are incorporated in their entirety to the extent not inconsistent with the teachings herein.

What is claimed is:

1. A pharmaceutical composition cytotoxic to cancer cells wherein said composition comprises a compound according to Formula I, or a pharmaceutically acceptable salt thereof, and a pharmaceutically acceptable carrier, wherein Formula I is: 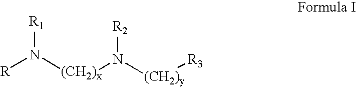
wherein R=anthracen-9-ylmethyl, R₁═H, R₂═H, R₃═NHR₄, x=4, and y=4; and wherein
R₄ is Et or Me, and wherein said compound is optionally a hydrochloride salt.

2. The composition of claim 1, wherein said compound is N-Anthracen-9-ylmethyl-N′-(4-ethylamino-butyl)-butane-1,4-diamine, Hydrochloride salt.

3. The composition of claim 1, wherein said compound is N-Anthracen-9-ylmethyl-N′-(4-methylamino-butyl)-butane-1,4-diamine, Hydrochloride salt.

4. A method of killing cancer cells in a patient in need thereof comprising administering a compound according to Formula I, or a pharmaceutically acceptable salt thereof,
wherein Formula I is: 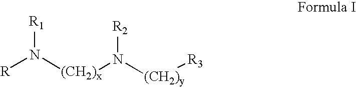
wherein R=anthracen-9-ylmethyl, R₁═H, R₂═H, R₃═NHR₄, x=4, and y=4; and wherein R₄ is Et or Me, and wherein said compound is optionally a hydrochloride salt.

5. The composition of claim 4, wherein said compound is N-Anthracen-9-ylmethyl-N′-(4-ethylamino-butyl)-butane-1,4-diamine, Hydrochloride salt.

6. The composition of claim 4, wherein said compound is N-Anthracen-9-ylmethyl-N-(4-methylamino-butyl)-butane-1,4-diamine, Hydrochloride salt.

7. A compound according to Formula I, or a pharmaceutically acceptable salt thereof,
wherein Formula I is: 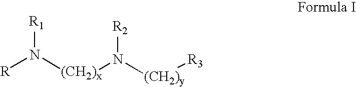
wherein R=anthracen-9-ylmethyl, R₁═H, R₂═H, R₃═NHR₄, x=4, and y=4; and wherein R₄ is Et or Me, and wherein said compound is optionally a hydrochloride salt.

8. The compound of claim 7, wherein said compound is N-Anthracen-9-ylmethyl-N′-(4-ethylamino-butyl)-butane-1,4-diamine, Hydrochloride salt.

9. The compound of claim 7, wherein said compound is N-Anthracen-9-ylmethyl-NA-(4-methylamino-butyl)-butane-1,4-diamine, Hydrochloride salt.
